(19) United States
(12) Patent Application Publication
(10) Pub. No.: US 2024/0047063 A1
(43) Pub. Date: Feb. 8, 2024

(54) ARTIFICIAL INTELLIGENCE-BASED SYSTEM AND PROCESS FOR PRECISION MEDICINE PRACTICE

(71) Applicant: RareAI Institute, Birmingham, AL (US)

(72) Inventor: Lurong Pan, Vestavia Hill, AL (US)

(21) Appl. No.: 17/818,034

(22) Filed: Aug. 8, 2022

(51) Int. Cl. G16H 50/20; G16H 70/40

(52) U.S. Cl. CPC G16H 50/20; G16H 70/40

(57) ABSTRACT

A system and method for drug discovery is disclosed. The method includes receiving patient multiomics raw data, extracting one or more reports from the patient multiomics raw data, and detecting one or more genome variants in the patient. Further, the method includes determining if the one or more genome variants are one or more known variants or one or more unknown variants, determining if the one or more genome variants are one or more coding variants or one or more non-coding variants, and determining if the patient is suffering from a functional loss or a functional excess. The method includes generating one or more rescue recommendations, determining one or more medical therapies for the patient, and outputting the one or more rescue recommendations and the one or more medical therapies to one or more electronic devices associated with the user.

### FIELD OF INVENTION

Embodiments of the present disclosure relate to Artificial Intelligence (AI)-based systems and more particularly relates to an AI-based system and method for drug discovery.

### BACKGROUND

Treating rare disease is a global challenge. It is often necessary for countries to work together to solve these issues. Rare diseases, which number over 8000 distinct illnesses, collectively affects over 300 million people in the whole world of which 200 million are children. These diseases contribute to the continued mortality of children. For example, rare diseases cause 35% of deaths in the first year of life and 30% of deaths of children before their 5th birthday of those that survive. Currently, the medical field can only diagnose 30% of rare diseases in patients. This conclusion is at odds with the current drug development process as it takes an average of 14 years and 2 billion dollars to develop a new therapeutic for a particular disease. Continued disinterest of pharma and biotech industries to develop therapies result in only 5% of rare disease groups having an FDA-approved treatment. Another related issue is “continued misdiagnosis”. On average, it takes 7.6 years in the U.S. and 5.6 years in the U.K. for correct diagnosis of a rare disease. During this time on average, a patient will often be misdiagnosed 2-3 times and sees four different physicians and four different specialists.

Recent advances in biomedical sciences have resulted in innovative drug treatments. Yet making these treatments available to consumers remains a challenge. On an average, it takes ten to fifteen years and 1.3 billion dollars to develop one new drug. The process of developing a new drug involves a complex system of testing in which even qualified candidates have a failure rate of 80%. The main reason for such complicated process is that biological systems involve a lot of complicated procedures. Conventionally, first principle-based models, such as molecular dynamics, quantum mechanical, molecular mechanical and the like are used to facilitate a drug discovery process. However, the conventional first principle-based models have multiple disadvantages, such as are treatments problem specific, scale varies, accuracy varies, expensive to compute, and the like. Further, deep learning artificial intelligence models are also used to facilitate the drug discovery process. However, the deep learning artificial intelligence models includes various disadvantages, such as uniformly applicable, over fitting due to data bias, expensive to train, low repeatability, and the like.

Hence, there is a need for an improved Artificial Intelligence (AI)-based computing system for drug discovery, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an Artificial Intelligence (AI)-based computing system for drug discovery is disclosed. The AI-based computing system includes one or more hardware processors and a memory coupled to the one or more hardware processors. The memory includes a plurality of modules in the form of programmable instructions executable by the one or more hardware processors. The plurality of modules include a patient data receiver module configured to receive patient multiomics raw data associated with a patient from one of: one or more electronic devices associated with a user and an external database. The plurality of modules include a data extracting module configured to extract one or more reports from the received patient multiomics raw data by using one or more data extraction techniques. The extracted one or more reports include a patient whole genome report, a patient exome report, and a patient Ribonucleic Acid (RNA) report. Further, the plurality of modules include a variant detection module configured to detect one or more genome variants in the patient by analyzing the extracted patient whole genome report. The one or more genome variants are permanent changes in a Deoxyribonucleic Acid (DNA) sequence that makes up a gene.

The plurality of modules also include a variant determination module configured to determine if the detected one or more genome variants are one of: one or more known variants and one or more unknown variants based on the extracted patient whole genome report and one or more diseases of the patient by using a variant determination-based Artificial Intelligence (AI) model. The plurality of modules includes a genome determination module configured to determine if the detected one or more genome variants are one of: one or more coding variants and one or more non-coding variants based on the extracted patient whole genome report by using a coding determination-based AI model upon determining that the detected one or more genome variants are the one or more unknown variants. Further, the plurality of modules includes a functional determination module configured to determine if the patient is suffering from one of: a functional loss and a functional excess by performing one or more analysis operations on the extracted one or more reports upon determining that the detected one or more genome variants are the one or more coding variants. The plurality of modules also include a data generation module configured to generate one or more rescue recommendations for the user to perform a functional rescue therapy on the patient based on result of the one or more analysis operations upon determining that the patient is suffering from the functional loss. Furthermore, the plurality of modules include a medical therapy recommendation module configured to determine one or more medical therapies for the patient based on result of the functional rescue therapy by using a functional rescue therapy-based AI model upon generating the one or more rescue recommendations. The one or more medical therapies correspond to a new drug discovery for one or more potential targets corresponding to the one or more coding variants of the patient. The plurality of modules include a data output module configured to output the generated one or more rescue recommendations and the determined one or more medical therapies on user interface screen of the one or more electronic devices associated with the user.

In accordance with another embodiment of the present disclosure, an AI-based method for drug discovery is disclosed. The AI-based method includes receiving patient multiomics raw data associated with a patient from one of: one or more electronic devices associated with a user and an external database. The AI-based method includes extracting one or more reports from the received patient multiomics raw data by using one or more data extraction techniques. The extracted one or more reports comprise a patient whole genome report, a patient exome report, and a patient RNA report. Further, the AI-based method includes detecting one or more genome variants in the patient by analyzing the extracted patient whole genome report. The one or more genome variants are permanent changes in a DNA sequence that makes up a gene. The AI-based method also includes determining if the detected one or more genome variants are one of: one or more known variants and one or more unknown variants based on the extracted patient whole genome report and one or more diseases of the patient by using a variant determination-based AI model. The AI-based method further includes determining if the detected one or more genome variants are one of: one or more coding variants and one or more non-coding variants based on the extracted patient whole genome report by using a coding determination-based AI model upon determining that the detected one or more genome variants are the one or more unknown variants. Further, the AI-based method includes determining if the patient is suffering from one of: a functional loss and a functional excess by performing one or more analysis operations on the extracted one or more reports upon determining that the detected one or more genome variants are the one or more coding variants. Furthermore, the AI-based method includes generating one or more rescue recommendations for the user to perform a functional rescue therapy on the patient based on result of the one or more analysis operations upon determining that the patient is suffering from the functional loss. The AI-based method includes determining one or more medical therapies for the patient based on result of the functional rescue therapy by using a functional rescue therapy-based AI model upon generating the one or more rescue recommendations. The one or more medical therapies correspond to a new drug discovery for one or more potential targets corresponding to the one or more coding variants of the patient. The AI-based method also includes outputting the generated one or more rescue recommendations and the determined one or more medical therapies on user interface screen of the one or more electronic devices associated with the user.

Embodiment of the present disclosure also provide a non-transitory computer-readable storage medium having instructions stored therein that, when executed by a hardware processor, cause the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for drug discovery, in accordance with an embodiment of the present disclosure;

FIG. 2 is a block diagram illustrating an exemplary Artificial Intelligence (AI)-based computing system for drug discovery, in accordance with an embodiment of the present disclosure;

FIG. 3 is a block diagram depicting an exemplary operation of the AI-based computing system, in accordance with an embodiment of the present disclosure;

FIG. 4 is a block diagram illustrating the AI-based computing system for drug discovery, in accordance with another embodiment of the present disclosure; and

FIG. 5 is a process flow diagram illustrating an exemplary AI-based method for drug discovery, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

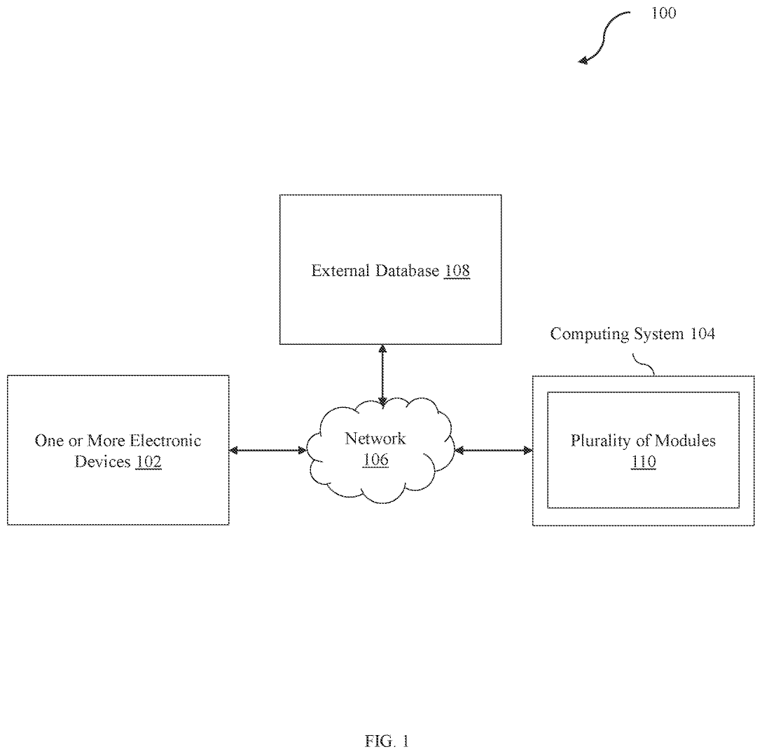

Referring now to the drawings, and more particularly to FIG. 1 through FIG. 5, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for drug discovery, in accordance with an embodiment of the present disclosure. According to FIG. 1, the computing environment 100 includes one or more electronic devices 102 associated with a user communicatively coupled to an Artificial intelligence (AI)-based computing system 104 via a network 106. In an exemplary embodiment of the present disclosure, the user may include a medical professional performing a drug discovery process. In an embodiment of the present disclosure, drug discovery is a process which aims at identifying a compound therapeutically useful in curing and treating disease. Further, the one or more electronic devices 102 are used by the user for providing patient multiomics raw data associated with a patient to the AI-based computing system 104. Furthermore, the one or more electronic devices 102 may also be used by the user to receive generated one or more rescue recommendations and determined one or more medical therapies from the AI-based computing system 104. The AI-based computing system 104 may be hosted on a central server, such as cloud server or a remote server. In an embodiment of the present disclosure, the AI-based computing system 104 may be a cloud-based virtual screening platform for drug candidate screening. Further, the network 106 may be a Wireless-Fidelity (Wi-Fi) connection, a hotspot connection, a Bluetooth connection, a local area network, a wide area network or any other wireless network. In an exemplary embodiment of the present disclosure, the one or more electronic devices 102 may include a laptop computer, desktop computer, tablet computer, smartphone, wearable device, smart watch, and the like.

Further, the computing environment 100 includes an external database 108 communicatively coupled to the AI-based computing system 104 via the network 106. The external database 108 stores the patient multiomics raw data. In an embodiment of the present disclosure, the external database 108 provides the patient multiomics raw data to the AI-based computing system 104.

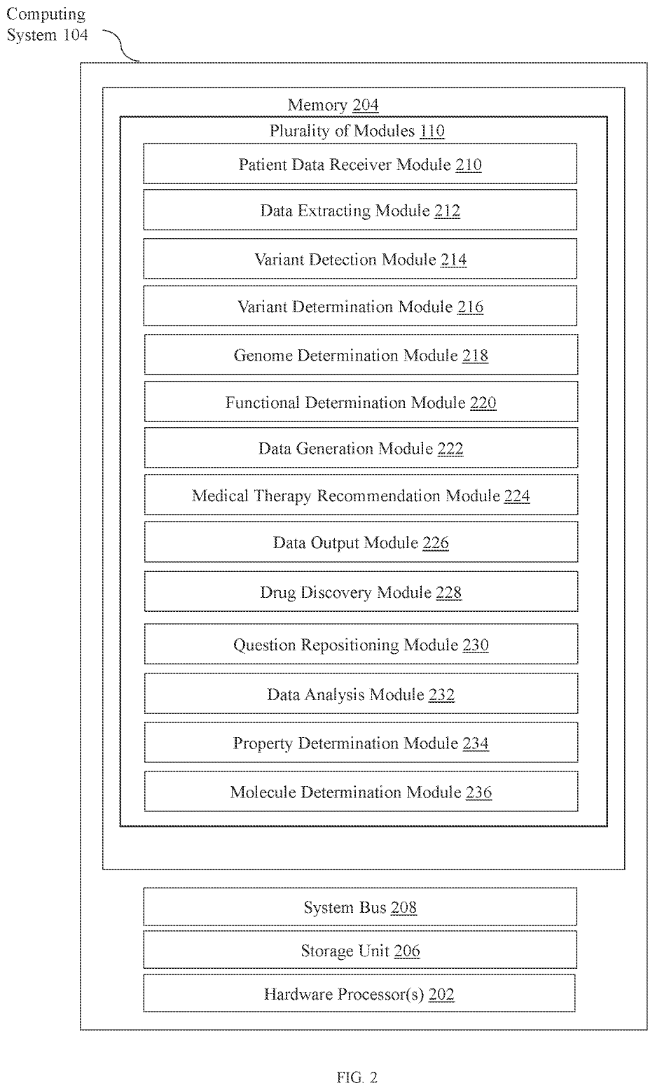

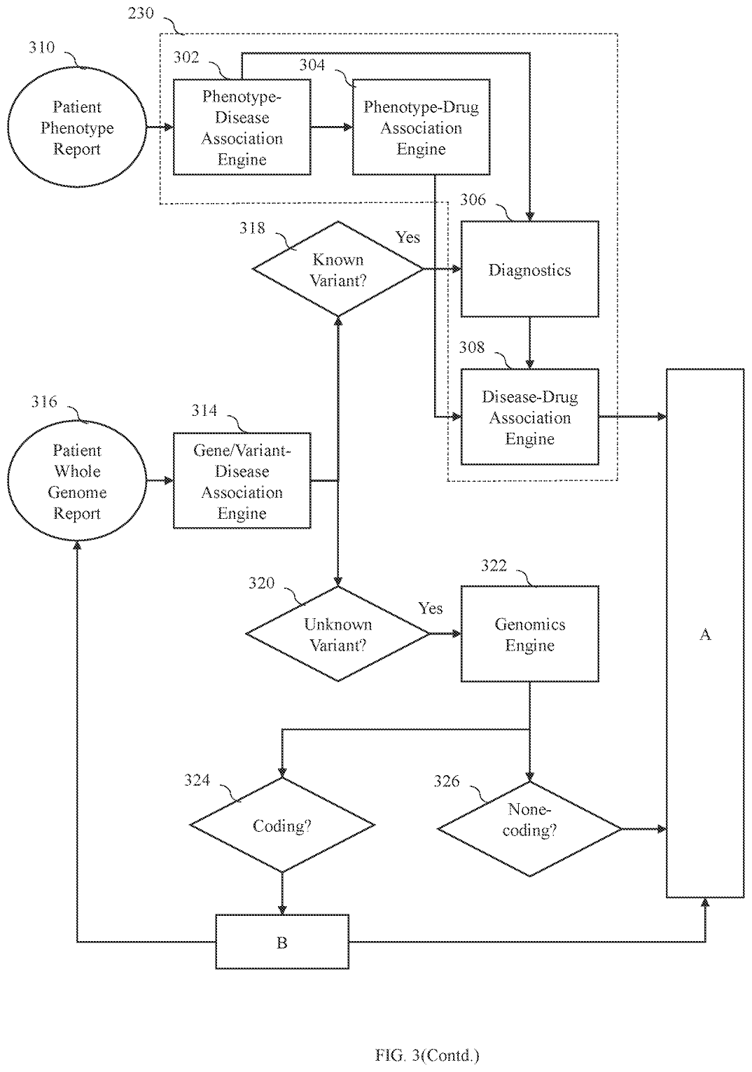

Furthermore, the one or more electronic devices 102 include a local browser, a mobile application or a combination thereof. Furthermore, the user may use a web application via the local browser, the mobile application, or a combination thereof to communicate with the AI-based computing system 104. In an embodiment of the present disclosure, the AI-based computing system 104 includes a plurality of modules 110. Details on the plurality of modules 110 have been elaborated in subsequent paragraphs of the present description with reference to FIG. 2.

In an embodiment of the present disclosure, the AI-based computing system 104 is configured to receive the patient multiomics raw data associated with the patient from the one or more electronic devices 102 associated with the user or the external database 108. The AI-based computing system 104 extracts one or more reports from the received patient multiomics raw data by using one or more data extraction techniques. In an embodiment of the present disclosure, the extracted one or more reports include a patient whole genome report, a patient exome report, and a patient Ribonucleic Acid (RNA) report. The AI-based computing system 104 detects one or more genome variants in the patient by analyzing the extracted patient whole genome report. Further, the AI-based computing system 104 determines if the detected one or more genome variants are one or more known variants, or one or more unknown variants based on the extracted patient whole genome report and one or more diseases of the patient by using a variant determination-based Artificial Intelligence (AI) model. The AI-based computing system 104 determines if the detected one or more genome variants are one or more coding variants, or one or more non-coding variants based on the extracted patient whole genome report by using a coding determination-based AI model upon determining that the detected one or more genome variants are the one or more unknown variants. The AI-based computing system 104 determines if the patient is suffering from a functional loss or a functional excess by performing one or more analysis operations on the extracted one or more reports upon determining that the detected one or more genome variants are the one or more coding variants. The AI-based computing system 104 generates one or more rescue recommendations for the user to perform a functional rescue therapy on the patient based on result of the one or more analysis operations upon determining that the patient is suffering from the functional loss. Furthermore, the AI-based computing system 104 determines one or more medical therapies for the patient based on result of the functional rescue therapy by using a functional rescue therapy-based AI model upon generating the one or more rescue recommendations. In an embodiment of the present disclosure, the one or more medical therapies correspond to a new drug discovery for one or more potential targets corresponding to the one or more coding variants of the patient. The AI-based computing system 104 outputs the generated one or more rescue recommendations and the determined one or more medical therapies on user interface screen of the one or more electronic devices 102 associated with the user.

FIG. 2 is a block diagram illustrating an exemplary AI-based computing system 104 for drug discovery, in accordance with an embodiment of the present disclosure. Further, the AI-based computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 110 in the form of programmable instructions executable by the one or more hardware processors 202. Further, the plurality of modules 110 includes a patient data receiver module 210, a data extracting module 212, a variant detection module 214, a variant determination module 216, a genome determination module 218, a functional determination module 220, a data generation module 222, a medical therapy recommendation module 224, a data output module 226, a drug discovery module 228, a drug repositioning module 230, a data analysis module 232, a property determination module 234, and a molecule determination module 236.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 110 stored in the form of machine-readable instructions on any of the above-mentioned storage media and may be in communication with and executed by the one or more hardware processors 202.

The storage unit 206 may be a cloud storage, a Structured Query Language (SQL) data store or a location on a file system directly accessible by the plurality of modules 110. The storage unit 206 may store the patient multiomics raw data, the one or more reports, the one or more genome variants, the one or more rescue recommendations, the one or more medical therapies, one or more inhibitor recommendations, one or more drug recommendations, one or more new drugs, one or more existing drugs, a patient phenotype report, one or more diseases of the patient, one or more medicines, one or more medicine recommendations, a molecular structure of one or more biomolecules, one or more properties of each of the one or more biomolecules, one or more desired properties, one or more novel molecules, and the like.

The patient data receiver module 210 is configured to receive the patient multiomics raw data associated with the patient from the one or more electronic devices 102 associated with the user or the external database 108. In an exemplary embodiment of the present disclosure, the user may include the medical professional performing the drug discovery process. In an embodiment of the present disclosure, the drug discovery is a process which aims at identifying a compound therapeutically useful in curing and treating disease. In an exemplary embodiment of the present disclosure, the one or more electronic devices 102 may include a laptop computer, desktop computer, tablet computer, smartphone, wearable device, smart watch, and the like.

The data extracting module 212 is configured to extract the one or more reports from the received patient multiomics raw data by using the one or more data extraction techniques. In an exemplary embodiment of the present disclosure, the extracted one or more reports include a patient whole genome report, a patient exome report, and a patient Ribonucleic Acid (RNA) report.

The variant detection module 214 is configured to detect the one or more genome variants in the patient by analyzing the extracted patient whole genome report. In an embodiment of the present disclosure, the one or more genome variants are permanent changes in a Deoxyribonucleic Acid (DNA) sequence that makes up a gene.

The variant determination module 216 is configured to determine if the detected one or more genome variants are the one or more known variants, or the one or more unknown variants based on the extracted patient whole genome report and the one or more diseases of the patient by using the variant determination-based Artificial Intelligence (AI) model.

The genome determination module 218 is configured to determine if the detected one or more genome variants are the one or more coding variants, or the one or more non-coding variants based on the extracted patient whole genome report by using the coding determination-based AI model upon determining that the detected one or more genome variants are the one or more unknown variants.

The functional determination module 220 is configured to determine if the patient is suffering from the functional loss or the functional excess by performing one or more analysis operations on the extracted one or more reports upon determining that the detected one or more genome variants are the one or more coding variants. In an embodiment of the present disclosure, the one or more analysis operations include an expression level check, a sequence based mutant analysis, a structural based mutant analysis, a functional impact analysis, or a combination thereof. In an embodiment of the present disclosure, the sequence based mutant analysis, the structure based mutant analysis, and functional impact analysis are performed on the patient exome report, and the expression level check and the functional impact analysis is performed on the patient RNA report to determine if the patient is suffering from the functional loss or the functional excess.

The data generation module 222 is configured to generate the one or more rescue recommendations for the user to perform the functional rescue therapy on the patient based on result of the one or more analysis operations upon determining that the patient is suffering from the functional loss.

The medical therapy recommendation module 224 is configured to determine the one or more medical therapies for the patient based on result of the functional rescue therapy by using a functional rescue therapy-based AI model upon generating the one or more rescue recommendations. In an embodiment of the present disclosure, the one or more medical therapies correspond to a new drug discovery for one or more potential targets corresponding to the one or more coding variants of the patient. In an exemplary embodiment of the present disclosure, the functional rescue therapy-based AI model is a communication dynamics-based AI model. For example, the one or more medical therapies include an activator therapy, a DNA therapy, an RNA therapy, a protein therapy, a cell therapy, or a combination thereof.

The data output module 226 is configured to output the generated one or more rescue recommendations and the determined one or more medical therapies on user interface screen of the one or more electronic devices 102 associated with the user.

In an embodiment of the present disclosure, the drug discovery module 228 generates one or more inhibitor recommendations for the user to perform an inhibitor therapy on the patient based on result of the one or more analysis operations upon determining that the patient is suffering from the functional excess. Further, the drug discovery module 228 determines if one or more drugs are available for one or more drug targets corresponding to the one or more coding variants based on result of the one or more analysis operations by using a target-drug association-based AI model upon generating the one or more inhibitor recommendations. Further, the drug discovery module 228 generates one or more drug recommendations for a known drug repositioning corresponding to the determined one or more drugs upon determining that the one or more drugs are available for the one or more drug targets. In an embodiment of the present disclosure, the generated one or more drug recommendations are outputted on user interface screen of the one or more electronic devices 102 associated with the user. The drug discovery module 228 identifies one or more new drugs for the one or more drug targets corresponding to the one or more coding variants based on result of the one or more analysis operations and the inhibitor therapy upon determining that the one or more drugs are not available for the one or more drug targets. Furthermore, the drug discovery module 228 determines one or more existing drugs having an ability to provide one or more therapeutic uses for the one or more drug targets corresponding to the one or more coding variants based on result of the one or more analysis operations and the inhibitor therapy by using a structure-based drug repurposing engine upon determining that the one or more drugs are not available for the one or more drug targets. The drug discovery module 228 generates one or more existing drug recommendations for an existing drug repositioning corresponding to the determined one or more existing drugs. In an embodiment of the present disclosure, the generated one or more existing drug recommendations are outputted on user interface screen of the one or more electronic devices 102 associated with the user.

Further, the drug repositioning module 230 receives a patient phenotype report of the patient from the one or more electronic devices 102 associated with the user. In an exemplary embodiment of the present disclosure, the patient phenotype report includes height, hair color, DNA sequence of the patient, and the like. The drug repositioning module 230 detects the one or more diseases of the patient based on the patient phenotype report, and International Classification of Diseases (ICD)-9 or ICD-10 by using a phenotype-disease association-based AI model. Furthermore, the drug repositioning module 230 determines one or more medicines for the detected one or more diseases based on the received patient phenotype report and one or more clinical guidelines by using a phenotype-drug association-based AI model upon determining that the detected one or more genome variants are the one or more known variants. The drug repositioning module 230 generates one or more medicine recommendations for a known medicine repositioning corresponding to the determined one or more medicines. In an embodiment of the present disclosure, the generated one or more medicine recommendations are outputted on user interface screen of the one or more electronic devices 102 associated with the user.

In an embodiment of the present disclosure, the data analysis module 232 performs a variant biopathway impact analysis on the extracted patient whole genome report upon determining that the one or more detected more genome variants are the one or more non-coding variants. Further, the data analysis module 232 determines if one or more drugs are available for one or more drug targets corresponding to the one or more non-coding variants based on result of the variant biopathway impact analysis by using a gene-drug association-based AI model upon performing the functional rescue therapy. The data analysis module 232 generates one or more drug recommendations for a known drug repositioning corresponding to the determined one or more drugs upon determining that the one or more drugs are available for the one or more drug targets. In an embodiment of the present disclosure, the generated one or more drug recommendations are outputted on user interface screen of the one or more electronic devices 102 associated with the user.

Furthermore, the data analysis module 232 identifies one or more new drugs for the one or more drug targets corresponding to the one or more non-coding variants based on result of the variant biopathway impact analysis upon determining that the one or more drugs are not available for the one or more non-coding variants. The data analysis module 232 determines one or more existing drugs having an ability to provide one or more therapeutic uses for the one or more drug targets corresponding to the one or more non-coding variants based on result of the variant biopathway impact analysis by using a pathway-based drug repurposing engine upon determining that the one or more drugs are not available for the one or more non-coding variants. The data analysis module 232 also generates one or more existing drug recommendations for a known drug repositioning corresponding to the determined one or more existing drugs. In an embodiment of the present disclosure, the generated one or more existing drug recommendations are outputted on user interface screen of the one or more electronic devices 102 associated with the user.

In an embodiment of the present disclosure, the property determination module 234 obtains a molecular structure of one or more biomolecules from the user. In an embodiment of the present disclosure, the molecular structure includes 1-Dimensional (D), 2D, 3D, or a combination thereof. For example, the one or more biomolecules include proteins, and nucleic acids. Further, the property determination module 234 determines one or more properties of each of the one or more biomolecules based on the obtained molecular structure of the one or more biomolecules by using a property determination-based AI model. In an exemplary embodiment of the present disclosure, the one or more properties include physical properties, chemical properties, biological properties, or a combination thereof.

Furthermore, the molecule determination module 236 obtains one or more desired properties of one or more biomolecules from the user. In an exemplary embodiment of the present disclosure, the one or more desired properties include desired physical properties, desired chemical properties, desired biological properties, or a combination thereof. The molecule determination module 236 is configured to determine one or more novel molecules based on the obtained one or more desired properties by using a molecule determination-based AI model.

In operation, the AI-based computing system 104 creates comprehensive knowledge maps of biological networks relevant to drug-biomarker interactions by integrating machine learning approaches and big data technology. The primary focus of modern biomedical science is to understand molecular level mechanisms and their pathological implications. However, the volume of data produced outstrips current analytical tools. The AI-based computing system 104 constructs comprehensive pathway knowledge maps based on public databases, using the entities (nodes) in databases as keys to perform data mining from research journals and patents. Next, we will develop a fully automated AI system to continually expand and update the maps. Once the map system is generated, probabilistic models can be developed and applied to build predictive models for drug discovery. With this computational resource, it is easier to understand disease models and predict potential drug candidates for potential drug targets. Further, the AI-based computing system 104 develops a cloud-based virtual screening platform for drug candidate screening using conventional computational physics and deep learning models. Machine learning techniques may be used within the framework of computational molecular design to increase accuracy. It may also be used independently as black-box models, trained by experimental data, to increase accuracy and decrease computational cost.

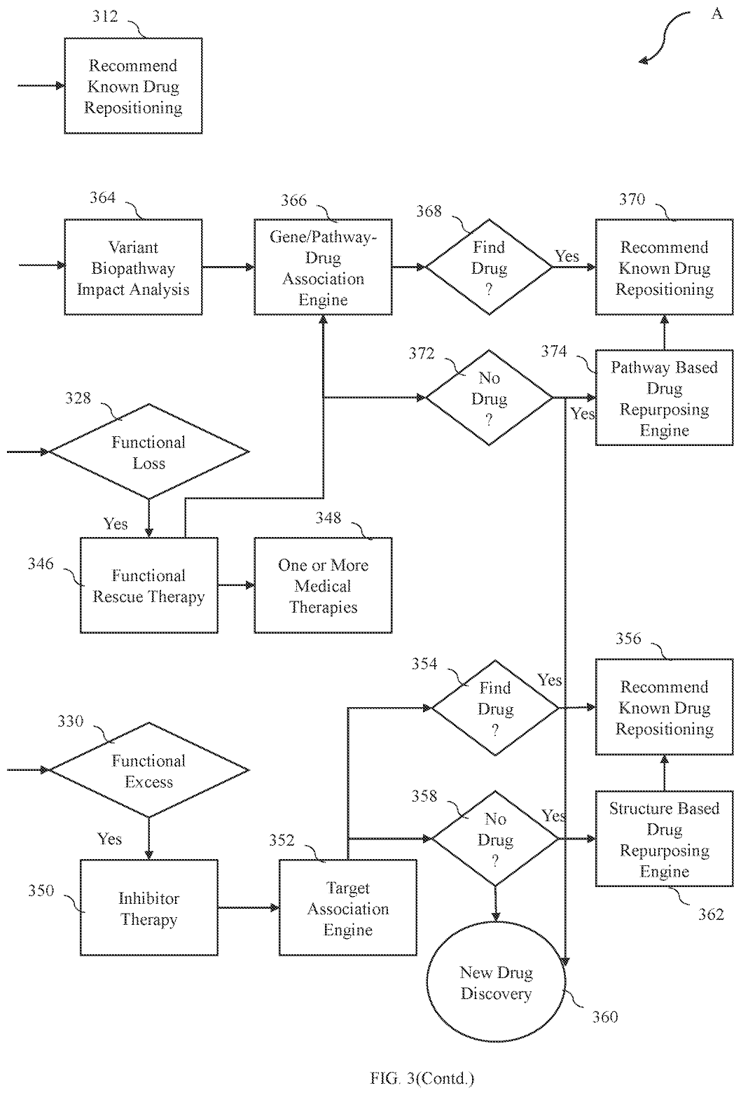

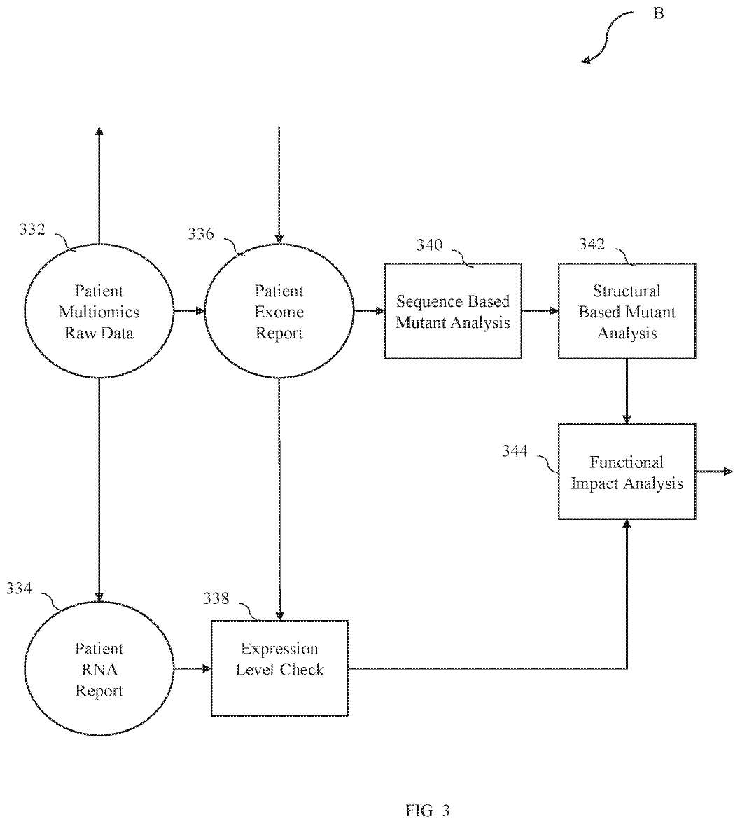

FIG. 3 is a block diagram depicting an exemplary operation of the AI-based computing system 104, in accordance with an embodiment of the present disclosure. The drug repositioning module 230 includes a phenotype-disease association engine 302, a phenotype-drug association engine 304, diagnostics 306, and a disease-drug association engine 308. In an embodiment of the present disclosure, the phenotype-disease association engine 302 receives the patient phenotype report 310 and perform the diagnostics 306 to detect the one or more diseases of the patient based on the patient phenotype report, and ICD-9 or ICD-10 by using the phenotype-disease association-based AI model. Further, the disease-drug association engine determines the one or more medicines for the detected one or more diseases based on the received patient phenotype report and the one or more clinical guidelines by using the phenotype-drug association-based AI model upon determining that the detected one or more genome variants are the one or more known variants. At 312, the one or more medicine recommendations are generated for a known medicine repositioning corresponding to the determined one or more medicines. In an embodiment of the present disclosure, the variant determination module 216 includes a gene/variant —disease association engine 314 configured to determine if the one or more genome variants are the one or more known variants or the one or more unknown variants based on the patient whole genome report 316 and the one or more diseases of the patient by using the variant determination-based AI model. At step 318, it is determined that the one or more genome variants are the one or more known variants. At step 320, it is determined that the one or more genome variants are the one or more unknown variants. Further, the genome determination module 218 includes a genomic engine 322 configured to determine if the detected one or more genome variants are the one or more coding variants 324 or the one or more non-coding variants 326 based on the patient whole genome report 316 by using the coding determination-based AI model upon determining that the detected one or more genome variants are the one or more unknown variants. The functional determination module 220 determines if the patient is suffering from the functional loss 328 or the functional excess 330 by performing the one or more analysis operations on the one or more reports upon determining that the detected one or more genome variants are the one or more coding variants 324. In an exemplary embodiment of the present disclosure, the one or more reports are the patient whole genome report 316, the patient multiomics raw data 332, the patient RNA report 334, and the patient exome report 336. For example, the one or more operations include the expression level check 338 which is performed on the patient RNA report 330. Further, the one or more operations include the sequence based mutant analysis 340, the structural based mutant analysis 342, and the functional impact analysis 344 which are performed on the patient exome report 336, and the patient multiomics raw data 332. The data generation module 222 generates the one or more rescue recommendations for the user to perform a functional rescue therapy 346 on the patient based on result of the one or more analysis operations upon determining that the patient is suffering from the functional loss 328. Further, the medical therapy recommendation module 224 determines the one or more medical therapies 348 for the patient based on result of the functional rescue therapy 346 by using the functional rescue therapy-based AI model upon generating the one or more rescue recommendations. The drug discovery module 228 generates the one or more inhibitor recommendations for the user to perform the inhibitor therapy 350 on the patient based on result of the one or more analysis operations upon determining that the patient is suffering from the functional excess 330. The drug discovery module 228 includes a target association engine 352 determine if the one or more drugs are available for one or more drug targets corresponding to the one or more coding variants based on result of the one or more analysis operations by using the target-drug association-based AI model upon generating the one or more inhibitor recommendations. At step 354, it is determined that the one or more drugs are available and at step 356, one or more drug recommendations are generated for the known drug repositioning corresponding to the determined one or more drugs. At step 358, it is determined that the one or more drugs are not available and at step 360, the one or more new drugs are discovered for the one or more drug targets corresponding to the one or more coding variants based on result of the one or more analysis operations and the inhibitor therapy 350. Further, the drug discovery module 228 includes a structure based drug repositioning engine 362 configured to determine the one or more existing drugs having an ability to provide one or more therapeutic uses for the one or more drug targets corresponding to the one or more coding variants based on result of the one or more analysis operations and the inhibitor therapy 350 by using the structure-based drug repurposing engine upon determining that the one or more drugs are not available for the one or more drug targets. At step 356, the one or more existing drug recommendations are generated for an existing drug repositioning corresponding to the determined one or more existing drugs.

Further, the data analysis module 232 is configured to perform the variant biopathway impact analysis 364 on the extracted patient whole genome report 316 upon determining that the one or more detected more genome variants are the one or more non-coding variants 326. The data analysis module 232 includes a gene/pathway drug association engine 366 configured to determine if one or more drugs are available for one or more drug targets corresponding to the one or more non-coding variants based on result of the variant biopathway impact analysis by using the gene-drug association-based AI model upon performing the functional rescue therapy 346. At step 368, it is determined that the one or more drugs are available and at step 370, the one or more drug recommendations are generated for a known drug repositioning corresponding to the determined one or more drugs. At step 372, it is determined that the one or more drugs are not available and at step 360, one or more new drugs are identified for the one or more drug targets corresponding to the one or more non-coding variants based on result of the variant biopathway impact analysis 364. The data analysis module 232 includes a pathway-based drug repurposing engine 374 configured to determine one or more existing drugs having an ability to provide one or more therapeutic uses for the one or more drug targets corresponding to the one or more non-coding variants based on result of the variant biopathway impact analysis by using the pathway-based drug repurposing engine upon determining that the one or more drugs are not available for the one or more non-coding variants. At step 370, one or more existing drug recommendations are generated for a known drug repositioning corresponding to the determined one or more existing drugs.

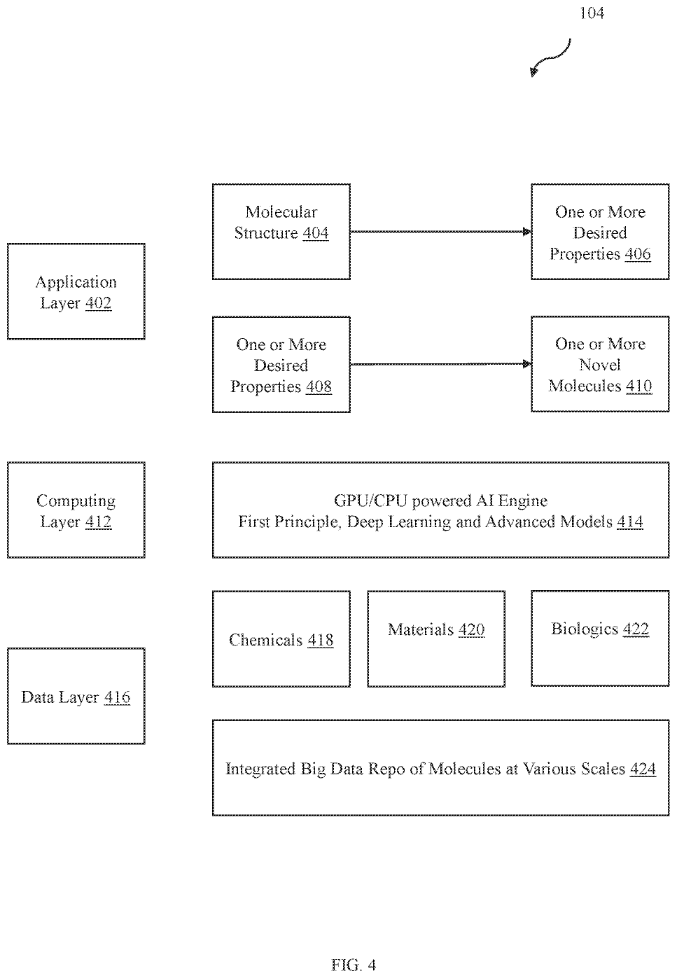

FIG. 4 is a block diagram illustrating the AI-based computing system 104 for drug discovery, in accordance with another embodiment of the present disclosure.

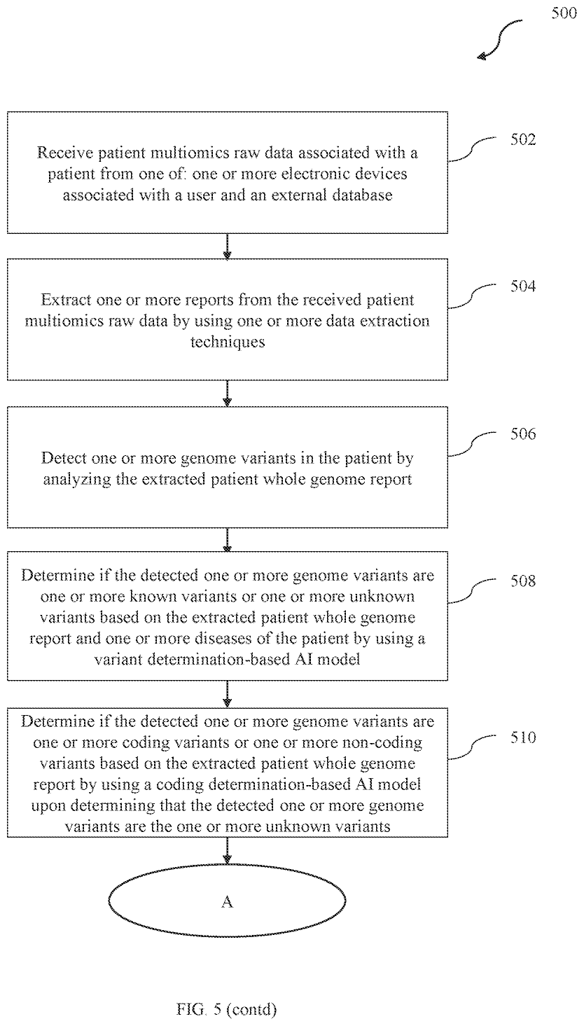

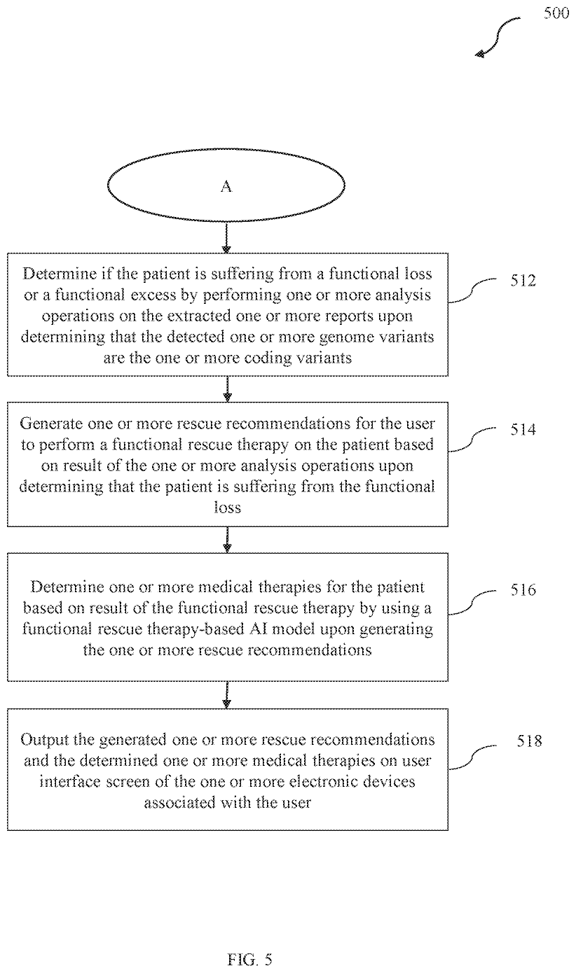

FIG. 5 is a process flow diagram illustrating an exemplary AI-based method 500 for drug discovery, in accordance with an embodiment of the present disclosure. At step 502, patient multiomics raw data associated with a patient is received from one or more electronic devices 102 associated with a user or an external database 108. In an exemplary embodiment of the present disclosure, the user may include a medical professional performing the drug discovery process. In an embodiment of the present disclosure, the drug discovery is a process which aims at identifying a compound therapeutically useful in curing and treating disease. In an exemplary embodiment of the present disclosure, the one or more electronic devices 102 may include a laptop computer, desktop computer, tablet computer, smartphone, wearable device, smart watch, and the like.

At step 504, one or more reports are extracted from the received patient multiomics raw data by using one or more data extraction techniques. In an exemplary embodiment of the present disclosure, the extracted one or more reports include a patient whole genome report, a patient exome report, and a patient RNA report.

At step 506, one or more genome variants are detected in the patient by analyzing the extracted patient whole genome report. In an embodiment of the present disclosure, the one or more genome variants are permanent changes in a DNA sequence that makes up a gene.

At step 508, it is determined if the detected one or more genome variants are one or more known variants, or one or more unknown variants based on the extracted patient whole genome report and the one or more diseases of the patient by using the variant determination-based AI model.

At step 510, it is determined if the detected one or more genome variants are one or more coding variants, or one or more non-coding variants based on the extracted patient whole genome report by using a coding determination-based AI model upon determining that the detected one or more genome variants are the one or more unknown variants.

At step 512, it is determined if the patient is suffering from a functional loss or a functional excess by performing one or more analysis operations on the extracted one or more reports upon determining that the detected one or more genome variants are the one or more coding variants. In an embodiment of the present disclosure, the one or more analysis operations include an expression level check, a sequence based mutant analysis, a structural based mutant analysis, a functional impact analysis, or a combination thereof. In an embodiment of the present disclosure, the sequence based mutant analysis, the structure based mutant analysis, and functional impact analysis are performed on the patient exome report, and the expression level check and the functional impact analysis is performed on the patient RNA report to determine if the patient is suffering from the functional loss or the functional excess.

At step 514, one or more rescue recommendations are generated for the user to perform the functional rescue therapy on the patient based on result of the one or more analysis operations upon determining that the patient is suffering from the functional loss.

At step 516, one or more medical therapies are determined for the patient based on result of the functional rescue therapy by using a functional rescue therapy-based AI model upon generating the one or more rescue recommendations. In an embodiment of the present disclosure, the one or more medical therapies correspond to a new drug discovery for one or more potential targets corresponding to the one or more coding variants of the patient. In an exemplary embodiment of the present disclosure, the functional rescue therapy-based AI model is a communication dynamics-based AI model. For example, the one or more medical therapies include an activator therapy, a DNA therapy, an RNA therapy, a protein therapy, a cell therapy, or a combination thereof.

At step 518, the generated one or more rescue recommendations and the determined one or more medical therapies are outputted on user interface screen of the one or more electronic devices 102 associated with the user.

In an embodiment of the present disclosure, the AI-based method 500 includes generating one or more inhibitor recommendations for the user to perform an inhibitor therapy on the patient based on result of the one or more analysis operations upon determining that the patient is suffering from the functional excess. Further, the AI-based method 500 includes determining if one or more drugs are available for one or more drug targets corresponding to the one or more coding variants based on result of the one or more analysis operations by using a target-drug association-based AI model upon generating the one or more inhibitor recommendations. Further, the AI-based method 500 includes generating one or more drug recommendations for a known drug repositioning corresponding to the determined one or more drugs upon determining that the one or more drugs are available for the one or more drug targets. In an embodiment of the present disclosure, the generated one or more drug recommendations are outputted on user interface screen of the one or more electronic devices 102 associated with the user. The AI-based method 500 includes identifying one or more new drugs for the one or more drug targets corresponding to the one or more coding variants based on result of the one or more analysis operations and the inhibitor therapy upon determining that the one or more drugs are not available for the one or more drug targets. Furthermore, the AI-based method 500 includes determining one or more existing drugs having an ability to provide one or more therapeutic uses for the one or more drug targets corresponding to the one or more coding variants based on result of the one or more analysis operations and the inhibitor therapy by using a structure-based drug repurposing engine upon determining that the one or more drugs are not available for the one or more drug targets. The AI-based method 500 includes generating one or more existing drug recommendations for an existing drug repositioning corresponding to the determined one or more existing drugs. In an embodiment of the present disclosure, the generated one or more existing drug recommendations are outputted on user interface screen of the one or more electronic devices 102 associated with the user.

Further, the AI-based method 500 includes receiving a patient phenotype report of the patient from the one or more electronic devices 102 associated with the user. In an exemplary embodiment of the present disclosure, the patient phenotype report includes height, hair color, DNA sequence of the patient, and the like. The AI-based method 500 includes detecting the one or more diseases of the patient based on the patient phenotype report, and International Classification of Diseases (ICD)-9 or ICD-10 by using a phenotype-disease association-based AI model. Furthermore, the AI-based method 500 includes determining one or more medicines for the detected one or more diseases based on the received patient phenotype report and one or more clinical guidelines by using a phenotype-drug association-based AI model upon determining that the detected one or more genome variants are the one or more known variants. The AI-based method 500 includes generating one or more medicine recommendations for a known medicine repositioning corresponding to the determined one or more medicines. In an embodiment of the present disclosure, the generated one or more medicine recommendations are outputted on user interface screen of the one or more electronic devices 102 associated with the user.

In an embodiment of the present disclosure, the AI-based method 500 includes performing a variant biopathway impact analysis on the extracted patient whole genome report upon determining that the one or more detected more genome variants are the one or more non-coding variants. Further, the AI-based method 500 includes determining if one or more drugs are available for one or more drug targets corresponding to the one or more non-coding variants based on result of the variant biopathway impact analysis by using a gene-drug association-based AI model upon performing the functional rescue therapy. The AI-based method 500 includes generating one or more drug recommendations for a known drug repositioning corresponding to the determined one or more drugs upon determining that the one or more drugs are available for the one or more drug targets. In an embodiment of the present disclosure, the generated one or more drug recommendations are outputted on user interface screen of the one or more electronic devices 102 associated with the user. Furthermore, the AI-based method 500 includes identifying one or more new drugs for the one or more drug targets corresponding to the one or more non-coding variants based on result of the variant biopathway impact analysis upon determining that the one or more drugs are not available for the one or more non-coding variants. The AI-based method 500 includes determining one or more existing drugs having an ability to provide one or more therapeutic uses for the one or more drug targets corresponding to the one or more non-coding variants based on result of the variant biopathway impact analysis by using a pathway-based drug repurposing engine upon determining that the one or more drugs are not available for the one or more non-coding variants. The AI-based method 500 includes generating one or more existing drug recommendations for a known drug repositioning corresponding to the determined one or more existing drugs. In an embodiment of the present disclosure, the generated one or more existing drug recommendations are outputted on user interface screen of the one or more electronic devices 102 associated with the user.

In an embodiment of the present disclosure, the AI-based method 500 includes obtaining a molecular structure of one or more biomolecules from the user. In an embodiment of the present disclosure, the molecular structure includes 1-Dimensional (D), 2D, 3D, or a combination thereof. For example, the one or more biomolecules include proteins, and nucleic acids. Further, the AI-based method 500 includes determining one or more properties of each of the one or more biomolecules based on the obtained molecular structure of the one or more biomolecules by using a property determination-based AI model. In an exemplary embodiment of the present disclosure, the one or more properties include physical properties, chemical properties, biological properties, or a combination thereof.

Furthermore, the AI-based method 500 includes obtaining one or more desired properties of one or more biomolecules from the user. In an exemplary embodiment of the present disclosure, the one or more desired properties include desired physical properties, desired chemical properties, desired biological properties, or a combination thereof. The AI-based method 500 includes determining one or more novel molecules based on the obtained one or more desired properties by using a molecule determination-based AI model.

The method 500 may be implemented in any suitable hardware, software, firmware, or combination thereof.

Thus, various embodiments of the present AI-based computing system 104 provide a solution to facilitate management of drug discovery. In an embodiment of the present disclosure, the AI-based computing system 104 uses communication dynamics powered AI. Thus, the AI-based computing system 104 includes multiple features, such as uniformly applicable, precision adjustable, least action principle for high learning efficiency, first principle, and the like. Further, the AI-based computing system 104 reduces complexity of the biological systems by means of an intelligent engine designed for the drug discovery process. In an embodiment of the present disclosure, a quickened ability to provide new drug treatments to patients would have local and global impact. Furthermore, the AI engine would be generalizable internationally. In an embodiment of the present disclosure, the AI-based computing system 104 makes drug development practical by radically shortening development and testing time. The proposed AI-based computing system 104 has the potential to screen 20 billion drug candidates in a few hours.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 208 to various devices such as a random-access memory (RAM), read-only memory (ROM), and an input/output (1/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

1. An Artificial Intelligence (AI)-based computing system for drug discovery, the computing system comprising:
one or more hardware processors; and
a memory coupled to the one or more hardware processors, wherein the memory comprises a plurality of modules in the form of programmable instructions executable by the one or more hardware processors, and wherein the plurality of modules comprises:
a patient data receiver module configured to receive patient multiomics raw data associated with a patient from one of: one or more electronic devices associated with a user and an external database;
a data extracting module configured to extract one or more reports from the received patient multiomics raw data by using one or more data extraction techniques, wherein the extracted one or more reports comprise a patient whole genome report, a patient exome report, and a patient Ribonucleic Acid (RNA) report;
a variant detection module configured to detect one or more genome variants in the patient by analyzing the extracted patient whole genome report, wherein the one or more genome variants are permanent changes in a Deoxyribonucleic Acid (DNA) sequence that makes up a gene;
a variant determination module configured to determine if the detected one or more genome variants are one of: one or more known variants and one or more unknown variants based on the extracted patient whole genome report and one or more diseases of the patient by using a variant determination-based Artificial Intelligence (AI) model;
a genome determination module configured to determine if the detected one or more genome variants are one of: one or more coding variants and one or more non-coding variants based on the extracted patient whole genome report by using a coding determination-based AI model upon determining that the detected one or more genome variants are the one or more unknown variants;
a functional determination module configured to determine if the patient is suffering from one of: a functional loss and a functional excess by performing one or more analysis operations on the extracted one or more reports upon determining that the detected one or more genome variants are the one or more coding variants;
a data generation module configured to generate one or more rescue recommendations for the user to perform a functional rescue therapy on the patient based on result of the one or more analysis operations upon determining that the patient is suffering from the functional loss;
a medical therapy recommendation module configured to determine one or more medical therapies for the patient based on result of the functional rescue therapy by using a functional rescue therapy-based AI model upon generating the one or more rescue recommendations, wherein the one or more medical therapies correspond to a new drug discovery for one or more potential targets corresponding to the one or more coding variants of the patient; and
a data output module configured to output the generated one or more rescue recommendations and the determined one or more medical therapies on user interface screen of the one or more electronic devices associated with the user.

2. The AI-based computing system of claim 1, further comprising a drug discovery module configured to:
generate one or more inhibitor recommendations for the user to perform an inhibitor therapy on the patient based on result of the one or more analysis operations upon determining that the patient is suffering from the functional excess;
determine if one or more drugs are available for one or more drug targets corresponding to the one or more coding variants based on result of the one or more analysis operations by using a target-drug association-based AI model upon generating the one or more inhibitor recommendations;
generate one or more drug recommendations for a known drug repositioning corresponding to the determined one or more drugs upon determining that the one or more drugs are available for the one or more drug targets, wherein the generated one or more drug recommendations are outputted on user interface screen of the one or more electronic devices associated with the user;
identify one or more new drugs for the one or more drug targets corresponding to the one or more coding variants based on result of the one or more analysis operations and the inhibitor therapy upon determining that the one or more drugs are not available for the one or more drug targets;
determine one or more existing drugs having an ability to provide one or more therapeutic uses for the one or more drug targets corresponding to the one or more coding variants based on result of the one or more analysis operations and the inhibitor therapy by using a structure-based drug repurposing engine upon determining that the one or more drugs are not available for the one or more drug targets; and
generate one or more existing drug recommendations for an existing drug repositioning corresponding to the determined one or more existing drugs, wherein the generated one or more existing drug recommendations are outputted on user interface screen of the one or more electronic devices associated with the user.

3. The AI-based computing system of claim 1, wherein the one or more medical therapies comprise at least one of: an activator therapy, a DNA therapy, an RNA therapy, a protein therapy and a cell therapy.

4. The AI-based computing system of claim 1, further comprising a drug repositioning module configured to:
receive a patient phenotype report of the patient from the one or more electronic devices associated with the user, wherein the patient phenotype report comprises height, hair color, and DNA sequence of the patient;
detect one or more diseases of the patient based on the patient phenotype report and one of: International Classification of Diseases (ICD)-9 and ICD-10 by using a phenotype-disease association-based AI model;
determine one or more medicines for the detected one or more diseases based on the received patient phenotype report and one or more clinical guidelines by using a phenotype-drug association-based AI model upon determining that the detected one or more genome variants are the one or more known variants; and
generate one or more medicine recommendations for a known medicine repositioning corresponding to the determined one or more medicines, wherein the generated one or more medicine recommendations are outputted on user interface screen of the one or more electronic devices associated with the user.

5. The AI-based computing system of claim 1, wherein the one or more analysis operations comprise at least one of: an expression level check, a sequence based mutant analysis, a structural based mutant analysis, and a functional impact analysis.

6. The AI-based computing system of claim 1, wherein the data analysis module is configured to:
perform a variant biopathway impact analysis on the extracted patient whole genome report upon determining that the one or more detected more genome variants are the one or more non-coding variants;
determine if one or more drugs are available for one or more drug targets corresponding to the one or more non-coding variants based on result of the variant biopathway impact analysis by using a gene-drug association-based AI model upon performing the functional rescue therapy;
generate one or more drug recommendations for a known drug repositioning corresponding to the determined one or more drugs upon determining that the one or more drugs are available for the one or more drug targets, wherein the generated one or more drug recommendations are outputted on user interface screen of the one or more electronic devices associated with the user;
identify one or more new drugs for the one or more drug targets corresponding to the one or more non-coding variants based on result of the variant biopathway impact analysis upon determining that the one or more drugs are not available for the one or more non-coding variants;
determine one or more existing drugs having an ability to provide one or more therapeutic uses for the one or more drug targets corresponding to the one or more non-coding variants based on result of the variant biopathway impact analysis by using a pathway-based drug repurposing engine upon determining that the one or more drugs are not available for the one or more non-coding variants; and
generate one or more existing drug recommendations for a known drug repositioning corresponding to the determined one or more existing drugs, wherein the generated one or more existing drug recommendations are outputted on user interface screen of the one or more electronic devices associated with the user.

7. The AI-based computing system of claim 1, wherein the functional rescue therapy-based AI model is a communication dynamics-based AI model.

8. The AI-based computing system of claim 1, further comprising a property determination module configured to:
obtain a molecular structure of one or more biomolecules from the user, wherein the molecular structure comprises at least one of: 1-Dimensional (D), 2D, and 3D, and wherein the one or more biomolecules comprise proteins, and nucleic acids; and
determine one or more properties of each of the one or more biomolecules based on the obtained molecular structure of the one or more biomolecules by using a property determination-based AI model, wherein the one or more properties comprise at least one of: physical properties, chemical properties and biological properties.

9. The AI-based computing system of claim 1, further comprising a molecule determination module configured to:
obtain one or more desired properties of one or more biomolecules from the user, wherein the one or more desired properties comprise at least one of: desired physical properties, desired chemical properties and desired biological properties; and
determine one or more novel molecules based on the obtained one or more desired properties by using a molecule determination-based AI model.

10. An Artificial Intelligence (AI)-based method for drug discovery, the method comprising:
receiving, by one or more hardware processors, patient multiomics raw data associated with a patient from one of: one or more electronic devices associated with a user and an external database;
extracting, by the one or more hardware processors, one or more reports from the received patient multiomics raw data by using one or more data extraction techniques, wherein the extracted one or more reports comprise a patient whole genome report, a patient exome report, and a patient Ribonucleic Acid (RNA) report;
detecting, by the one or more hardware processors, one or more genome variants in the patient by analyzing the extracted patient whole genome report, wherein the one or more genome variants are permanent changes in a Deoxyribonucleic Acid (DNA) sequence that makes up a gene;
determining, by the one or more hardware processors, if the detected one or more genome variants are one of: one or more known variants and one or more unknown variants based on the extracted patient whole genome report and one or more diseases of the patient by using a variant determination-based Artificial Intelligence (AI) model;
determining, by the one or more hardware processors, if the detected one or more genome variants are one of: one or more coding variants and one or more non-coding variants based on the extracted patient whole genome report by using a coding determination-based AI model upon determining that the detected one or more genome variants are the one or more unknown variants;
determining, by one or more hardware processors, if the patient is suffering from one of: a functional loss and a functional excess by performing one or more analysis operations on the extracted one or more reports upon determining that the detected one or more genome variants are the one or more coding variants;
generating, by one or more hardware processors, one or more rescue recommendations for the user to perform a functional rescue therapy on the patient based on result of the one or more analysis operations upon determining that the patient is suffering from the functional loss;
determining, by the one or more hardware processors, one or more medical therapies for the patient based on result of the functional rescue therapy by using a functional rescue therapy-based AI model upon generating the one or more rescue recommendations, wherein the one or more medical therapies correspond to a new drug discovery for one or more potential targets corresponding to the one or more coding variants of the patient; and
outputting, by the one or more hardware processors, the generated one or more rescue recommendations and the determined one or more medical therapies on user interface screen of the one or more electronic devices associated with the user.

11. The AI-based method of claim 10, further comprising:
generating one or more inhibitor recommendations for the user to perform an inhibitor therapy on the patient based on result of the one or more analysis operations upon determining that the patient is suffering from the functional excess;
determining if one or more drugs are available for one or more drug targets corresponding to the one or more coding variants based on result of the one or more analysis operations by using a target-drug association-based AI model upon generating the one or more inhibitor recommendations;
generating one or more drug recommendations for a known drug repositioning corresponding to the determined one or more drugs upon determining that the one or more drugs are available for the one or more drug targets, wherein the generated one or more drug recommendations are outputted on user interface screen of the one or more electronic devices associated with the user;
identifying one or more new drugs for the one or more drug targets corresponding to the one or more coding variants based on result of the one or more analysis operations and the inhibitor therapy upon determining that the one or more drugs are not available for the one or more drug targets;
determining one or more existing drugs having an ability to provide one or more therapeutic uses for the one or more drug targets corresponding to the one or more coding variants based on result of the one or more analysis operations and the inhibitor therapy by using a structure-based drug repurposing engine upon determining that the one or more drugs are not available for the one or more drug targets; and
generating one or more existing drug recommendations for an existing drug repositioning corresponding to the determined one or more existing drugs, wherein the generated one or more existing drug recommendations are outputted on user interface screen of the one or more electronic devices associated with the user.

12. The AI-based method of claim 10, wherein the one or more medical therapies comprise at least one of: an activator therapy, a DNA therapy, an RNA therapy, a protein therapy and a cell therapy.

13. The AI-based method of claim 10, further comprising:
receiving a patient phenotype report of the patient from the one or more electronic devices associated with the user, wherein the patient phenotype report comprises height, hair color, and DNA sequence of the patient;
detecting one or more diseases of the patient based on the patient phenotype report and one of: International Classification of Diseases (ICD)-9 and ICD-10 by using a phenotype-disease association-based AI model;
determining one or more medicines for the detected one or more diseases based on the received patient phenotype report and one or more clinical guidelines by using a phenotype-drug association-based AI model upon determining that the detected one or more genome variants are the one or more known variants; and
generating one or more medicine recommendations for a known medicine repositioning corresponding to the determined one or more medicines, wherein the generated one or more medicine recommendations are outputted on user interface screen of the one or more electronic devices associated with the user.

14. The AI-based method of claim 10, wherein the one or more analysis operations comprise at least one of: an expression level check, a sequence based mutant analysis, a structural based mutant analysis, and a functional impact analysis.

15. The AI-based method of claim 10, further comprising:
performing a variant biopathway impact analysis on the extracted patient whole genome report upon determining that the one or more detected more genome variants are the one or more non-coding variants;
determining if one or more drugs are available for one or more drug targets corresponding to the one or more non-coding variants based on result of the variant biopathway impact analysis by using a gene-drug association-based AI model upon performing the functional rescue therapy;
generating one or more drug recommendations for a known drug repositioning corresponding to the determined one or more drugs upon determining that the one or more drugs are available for the one or more drug targets, wherein the generated one or more drug recommendations are outputted on user interface screen of the one or more electronic devices associated with the user;
identifying one or more new drugs for the one or more drug targets corresponding to the one or more non-coding variants based on result of the variant biopathway impact analysis upon determining that the one or more drugs are not available for the one or more non-coding variants;
determining one or more existing drugs having an ability to provide one or more therapeutic uses for the one or more drug targets corresponding to the one or more non-coding variants based on result of the variant biopathway impact analysis by using a pathway-based drug repurposing engine upon determining that the one or more drugs are not available for the one or more non-coding variants; and
generating one or more existing drug recommendations for a known drug repositioning corresponding to the determined one or more existing drugs, wherein the generated one or more existing drug recommendations are outputted on user interface screen of the one or more electronic devices associated with the user.

16. The AI-based method of claim 10, wherein the functional rescue therapy-based AI model is a communication dynamics-based AI model.

17. The AI-based method of claim 10, further comprising:
obtaining a molecular structure of one or more biomolecules from the user, wherein the molecular structure comprises at least one of: 1-Dimensional (D), 2D, and 3D, and wherein the one or more biomolecules comprise proteins, and nucleic acids; and
determining one or more properties of each of the one or more biomolecules based on the obtained molecular structure of the one or more biomolecules by using a property determination-based AI model, wherein the one or more properties comprise at least one of: physical properties, chemical properties and biological properties.

18. The AI-based method of claim 10, further comprising:
obtaining one or more desired properties of one or more biomolecules from the user, wherein the one or more desired properties comprise at least one of: desired physical properties, desired chemical properties and desired biological properties; and
determining one or more novel molecules based on the obtained one or more desired properties by using a molecule determination-based AI model.

19. A non-transitory computer-readable storage medium having instructions stored therein that, when executed by a hardware processor, cause the processor to perform method steps comprising:
receiving patient multiomics raw data associated with a patient from one of: one or more electronic devices associated with a user and an external database;
extracting one or more reports from the received patient multiomics raw data by using one or more data extraction techniques, wherein the extracted one or more reports comprise a patient whole genome report, a patient exome report, and a patient Ribonucleic Acid (RNA) report;
detecting one or more genome variants in the patient by analyzing the extracted patient whole genome report, wherein the one or more genome variants are permanent changes in a Deoxyribonucleic Acid (DNA) sequence that makes up a gene;
determining if the detected one or more genome variants are one of: one or more known variants and one or more unknown variants based on the extracted patient whole genome report and one or more diseases of the patient by using a variant determination-based Artificial Intelligence (AI) model;
determining if the detected one or more genome variants are one of: one or more coding variants and one or more non-coding variants based on the extracted patient whole genome report by using a coding determination-based AI model upon determining that the detected one or more genome variants are the one or more unknown variants;
determining if the patient is suffering from one of; a functional loss and a functional excess by performing one or more analysis operations on the extracted one or more reports upon determining that the detected one or more genome variants are the one or more coding variants;
generating one or more rescue recommendations for the user to perform a functional rescue therapy on the patient based on result of the one or more analysis operations upon determining that the patient is suffering from the functional loss;
determining one or more medical therapies for the patient based on result of the functional rescue therapy by using a functional rescue therapy-based AI model upon generating the one or more rescue recommendations, wherein the one or more medical therapies correspond to a new drug discovery for one or more potential targets corresponding to the one or more coding variants of the patient; and
outputting the generated one or more rescue recommendations and the determined one or more medical therapies on user interface screen of the one or more electronic devices associated with the user.

20. The non-transitory computer-readable storage medium of claim 19, wherein the one or more analysis operations comprise at least one of: an expression level check, a sequence based mutant analysis, a structural based mutant analysis, and a functional impact analysis.
